(19) United States
(12) Patent Application Publication
(10) Pub. No.: US 2008/0051805 A1
(43) Pub. Date: Feb. 28, 2008

(54) Hair Follicle Coring Tool and System Based Thereon

(76) Inventor: Leonard Pinchuk, Miami, FL (US)

Correspondence Address: GORDON & JACOBSON, P.C., 60 LONG RIDGE ROAD, SUITE 407, STAMFORD, CT, 06902, US

(21) Appl. No.: 11/467,733

(22) Filed: Aug. 28, 2006

(51) Int. Cl. A61B 17/50

(52) U.S. Cl. 606/133

(57) ABSTRACT

A tool for coring a portion of one or more hair follicles incorporates an elongate hollow tubular member with at least one cut-out disposed between its distal end and proximal end. The cut-out allows for removal of debris that moves it way up the hollow tubular member from the opened distal tip. The needle can also include a radially-extending projection that cooperates with zero or more ring-shaped members that are slipped concentrically over the distal end of the hollow tubular member to set the penetration depth of the coring needle. The tool can also include a stepped interface that is adapted to snag a root of a hair follicle unit cored by the hollow tubular member upon its withdrawal away from the patient's body.

### BACKGROUND OF THE INVENTION

1. Field of the Invention

This invention relates to methods and apparatus for coring hair follicles (or portions thereof) for the purpose of transplantation into areas where improved hair growth is desired.

2. State of the Art

A hair follicle is a tiny tubular structure in the skin, contiguous with the top skin layer, or epidermis, that includes a tubular canal, a bulb (or vestibule) within the dermis skin layer, one or more hair shafts, a sheath that surrounds the lower part (root) of the hair shaft(s), the sebaceous (oil) gland, and the muscle anchored to the follicle's side wall. Autologous hair transplants remove the patient's own hair follicles (or portions thereof) from one or more donor area(s) where there is relatively thick hair growth. The removed hair follicles (or portions thereof) are then implanted to one or more areas where improved hair growth is desired.

The traditional method for removing hair follicles in autologous hair transplants removes a patch of skin from the donor area, which is typically located in the back of the patient's head. The patch is selected so as to contain a plurality of hair follicles where each hair follicle consists, in the majority of cases, of a root plus one to three hairs. The patch is placed on ice to maintain it cool and hydrated in saline so as to not kill tissue. The patch is subsequently placed under a microscope where a technician slices and dissects individual hair follicles from the patch. Each discrete hair follicle is then implanted in an area of the head where hair growth is desired. The problems with this technique include scarring (i.e., the skin area where the patch is removed scars), and pain (i.e., removal of the patch is painful). Moreover, the area where the patch of skin was removed is devoid of hair and thus is cosmetically unappealing.

A new technique cores out hair follicle units directly from the donor area. This technique is advantageous because it is less painful than the patch removal technique and the scar that may form is very small and difficult to see. The hair follicle unit consists of the hair shaft, sheath, and root of an individual hair follicle structure. A coring device is placed around groups of one to four hairs removing an average of 50 percent of the intact hair follicles of the group. Therefore, an average of 50 percent of the hair follicles of the group are left behind to grow. Additionally, only 50-60 percent of the hair follicles in the donor area are selected for coring, with the remaining percentage of hair follicles being left alone with no intervention. In this manner, any decrease in the visible density of hair in the donor area (about 30% on average) is virtually undetectable to the naked eye, which makes this new technique more cosmetically appealing than the patch removal technique.

The tool normally used to core out the hair follicle units is a hollow coring needle (or punch) held by a pin vice handle. The needle tip is typically realized from titanium nitride and has a diameter between 0.5 and 2.0 mm. The needle tip can come in different shapes (circle, concave, square, etc.). The physician first cuts the patient's hair in the donor area to the point where the hair is less than 2 mm long, and then the hollow coring needle is placed over a hair follicle unit and manually rotated by finger movements while pressing gently downward. The manual rotation is performed in a repeating clockwise and counterclockwise manner until the hair follicle unit is separated from the surrounding skin. This coring process is repeated for a multitude of hair follicle units. Periodically, the cored-out hair follicle units are removed from the head with forceps and placed in a saline solution on ice. The hair follicle units are then prepared and transplanted in the usual manner. It is not unusual for the patient to require transplantation of 1,200 or more hair follicle units over a prolonged period of time, which may be six hours or more.

Practitioners of this technique complain that the coring needles usually last for the coring of only 500 to 1,000 hair follicle grafts before becoming dull. The common range is 700 to 1,000 grafts, and sometimes longer. The coring needles as well as the pin vice handle are also required to be sterilized for each use. It is also difficult to control penetration depth of the needle tip into the scalp. Finally, it is common for debris to become stuck in the needle, which stops the coring procedure. A wire or smaller needle must then be inserted into the lumen of the coring needle to clean out the debris. Stopping the procedure and cleaning the needle is time consuming as well as potentially damaging to the needle as the wire that is inserted into the coring needle can chip the sharp end of the needle and cause it to be blunt.

### SUMMARY OF THE INVENTION

It is therefore an object of the invention to provide a tool for coring hair follicles (or portions thereof) that require less cleaning and maintenance during the coring process as compared to prior art designs.

It is a further object of the invention to provide such a tool that is pre-sterilized and disposable.

It is another object of the invention to provide such a tool whose penetration depth into the body can be effectively and efficiently controlled by the user.

It is yet another object of the invention to provide such a tool that can core and remove an individual hair follicle portion in one operation without the use of forceps or other tissue gripping mechanisms.

In accord with these objects, which will be discussed in detail below, a hair follicle coring tool is provided that includes an elongate hollow tubular member with at least one cut-out disposed between its distal end and proximal end. The cut-out allows for removal of debris that moves its way up the hollow tubular member from the opened distal tip. The needle can also include a radially-extending projection that cooperates with zero or more ring-shaped members that are slipped concentrically over the distal end of the hollow tubular member to set the penetration depth of the coring needle.

In one embodiment of the invention, the tool includes a stepped interface that is adapted to snag a root of a hair follicle unit cored by the hollow tubular member upon its withdrawal from the patient's body.

The tool is preferably realized from stainless steel coated with titanium nitride, zirconium, diamond tipped stainless steel, or any other material commonly used for needles, knives, drills and other tooling that is required to be sharp for prolonged periods of use. In disposable applications, the tool is packaged in a sterilized manner, which avoids the need for the practitioner to sterilize the tool before use.

In another aspect, an apparatus is provided for maintaining hair follicle portions cold and hydrated. The apparatus includes a fluid circulation loop carrying cold circulating fluid, and a filter that captures hair follicle portions carried by the cold circulating fluid.

Additional objects and advantages of the invention will become apparent to those skilled in the art upon reference to the detailed description taken in conjunction with the provided figures.

### BRIEF DESCRIPTION OF THE DRAWINGS

FIG. 1 is a perspective view of a coring needle for coring an individual hair follicle unit from a patient's scalp in accordance with the present invention.

FIG. 2 is a schematic side view of the coring needle of FIG. 1.

FIG. 3 is a schematic side view of a coring needle for coring an individual hair follicle unit from a patient's scalp in accordance with a second embodiment of the present invention.

FIG. 4 is a schematic side view of a coring needle for coring an individual hair follicle unit from a patient's scalp in accordance with a third embodiment of the present invention.

FIGS. 5A-5C are a sequence of schematic diagrams that illustrate a method of coring an individual hair follicle unit using the coring needle of FIG. 4.

FIG. 6 is a schematic side view of a coring needle for coring an individual hair follicle unit from a patient's scalp in accordance with a fourth embodiment of the present invention.

FIG. 7 is a schematic diagram illustrating a method of coring an individual hair follicle unit with the coring needle of FIG. 6.

FIG. 8 is a schematic diagram of an apparatus for maintaining cored hair follicle units cool and hydrated for autologous hair transplantation.

### DETAILED DESCRIPTION OF THE PREFERRED EMBODIMENTS

As used herein, the term “distal” is generally defined as in the direction of the patient, or away from a user of the device. Conversely, “proximal” generally means in the direction away from the patient, or toward the user of the device. A “hair follicle unit” consists of the hair shaft, sheath, and root of an individual hair follicle structure.

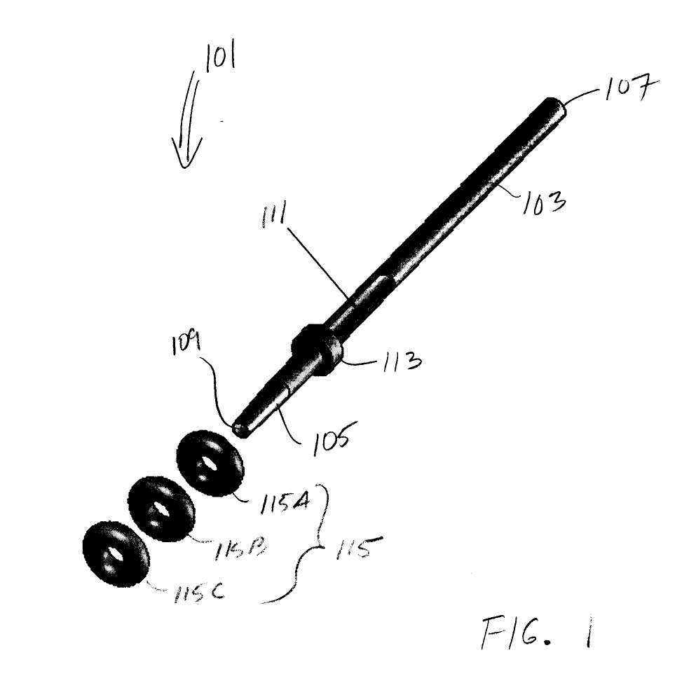

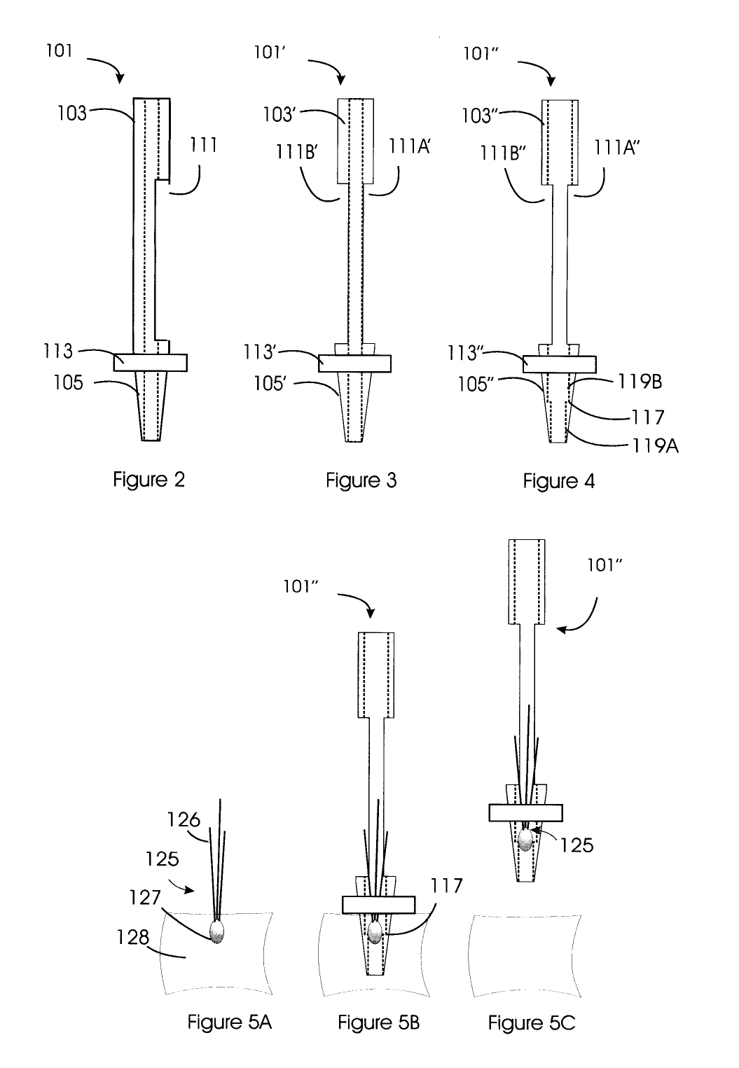

Turning now to FIGS. 1 and 2, there is shown an illustrative embodiment of a coring needle 101 of the present invention, which is used to core an individual hair follicle unit from a donor area of the patient's scalp for transplantation to an area where improved hair growth is desired. The coring needle 101 includes a hollow tubular member 103 with a distal end 105 opposite a proximal end 107. Preferably, the distal end 105 is sharpened, while the proximal end 107 is blunt as shown. The tubular member 103 is constructed such that it is substantially rigid in response to the forces applied thereto during use in coring a hair follicle unit. In the preferred embodiment, the tubular member 103 has an inside diameter in the range between 0.3 mm and 1.2 mm (most preferably on the order of 0.7 to 1.0 mm) and an outside diameter in the range between 0.7 mm and 1.5 mm (most preferably on the order of 0.75 to 1.2 mm), and the sharpened opened tip 109 defines an opening with a diameter in the range between 0.5 mm and 1.2 mm (most preferably on the order of 0.75 to 1.0 mm). Such diameters enable the physician to core an individual hair follicle unit with minimal transection. Other diameters can be used. Note that larger diameters provide a decreased transection rate, but more noticeable scaring.

A cut-out (or slot) 111 is formed in the annular wall of the tubular member 103 between the two ends 105 and 107. Preferably, the cut-out 111 has a major dimension that extends parallel to the central axis of the tubular member 103 and a minor dimension that extends substantially orthogonal relative to the central axis of the tubular member 103 as shown in FIGS. 1 and 2. The cut-out 111 provides an opening for the removal of debris that may work its way up the coring needle 101 during use. Such debris typically falls from the cut-out 111 on its own, which minimizes clogging of the tubular member 103 with limited human intervention. In the preferred embodiment, the distal end of the cut-out 111 is disposed 1 to 6 mm from the opened tip 109. In this configuration, debris must travel a short 1 to 6 mm length before it reaches the cut-out 111 where it generally falls out.

A stopper ring 113 extends radially outward from the hollow tubular member 103 and is disposed between the cut-out 111 and the opened tip 109. Preferably, the stopper ring 113 is welded, braised, swaged, or glued to hollow tubular member 103. The stopper ring 113 is used with zero or more O-rings 115 (three shown as 115A, 115B, 115C) to set the maximum depth of penetration of the coring needle 101 into the scalp. The O-rings 115 are slipped concentrically over the sharpened distal end 105 of the coring needle 101. The first O-ring 115A rests against the stopper ring 113. If more than one O-ring is used, the additional O-rings rest against each other. The distance between the distal-most surface of the O-rings 115 (or the distal-most surface of the stopper ring 113 if no O-rings are used) and the open tip 109 of the coring needle 101 defines the maximum depth of penetration of the coring needle 101 into the scalp during use. The stopper ring 113 is preferably located less than 10 mm from the opened tip 109. In the preferred configuration, the stopper ring 113 is located on the order of 5 mm from the opened tip 109 and the O-rings 115 are the order of 1 mm in thickness. In this configuration, zero O-rings are used to core a 5 mm deep hole, one O-ring is used to core a 4 mm deep hole, two O-rings are used to core a 3 mm deep hole, and three O-rings are used to core a 2 mm hole, etc. Note that the position of the stopper ring 113 and the thicknesses and lengths of the O-ring spacers can be varied to support a wide range of coring depths. O-rings can be made from Viton, SS, Teflon, Polyurethane, Nylon, Polyethylene, PP, silicone rubber, and the like.

The coring needle 101 is preferably realized from stainless steel and an anti-wear coating (such as a titanium nitride coating, a zirconium coating, or a diamond coating). Alternatively, the coring needing 101 can be realized from any other material commonly used for needles, knives, drills and other tooling that is required to be sharp for prolonged periods of use. In disposable applications, one or more coring needles 101 as well a number of O-ring spacers 115 (if any) are packaged in a sterilized manner, which avoids the need for the practitioner to sterilize such parts before use.

The coring needle 101 can be grasped with any suitable pin vice (or other support structure) and used, with and without twisting, in order to core a hair follicle unit from a donor area of the patient's scalp for transplantation to an area where improved hair growth is desired.

A coring needle 101′ in accordance with a second embodiment of the invention is shown in FIG. 3. The coring needle 101′ includes a hollow tubular member 103′ with two slots 111A′, 111B′ that are disposed opposite one another in the annular wall of the tubular member 103′. Preferably, the slots 111A′, 111B′ each have a major dimension that extends parallel to the central axis of the tubular member 103′ and a minor dimension that extends substantially orthogonal relative to the central axis of the tubular member 103′ as is evident in FIG. 3. Preferably, the distal end 105′ is sharpened, while the proximal end 107′ is blunt. The tubular member 103′ is constructed such that it is substantially rigid in response to the forces applied thereto during use in coring a hair follicle unit. A stopper ring 113′ extends radially outward from the hollow tubular member 103′ and is disposed between the cut-outs 111A′, 111B′ and the opened tip. The stopper ring 113′ is used with zero or more O-rings (not shown) to set the maximum depth of penetration of the coring needle 101′ into the scalp.

A coring needle 101″ in accordance with a third embodiment of the invention is shown in FIG. 4. The coring needle 101″ includes a hollow tubular member 103″ with two slots 111A″, 111B″ that are disposed opposite one another in the annular wall of the tubular member 103″. Preferably, the slots 111A″, 111B″ each have a major dimension that extends parallel to the central axis of the tubular member 103″ and a minor dimension that extends substantially orthogonal relative to the central axis of the tubular member 103″ as is evident in FIG. 4. Preferably, the distal end 105″ is sharpened, while the proximal end 101″ is blunt. The tubular member 103″ is constructed such that it is substantially rigid in response to the forces applied thereto during use in coring a hair follicle unit. A stopper ring 113″ extends radially outward from the hollow tubular member 103″ and is disposed between the cut-outs 111A″, 111B″ and the opened tip. The stopper ring 113″ is used with zero or more O-rings (not shown) to set the maximum depth of penetration of the coring needle 101″ into the scalp. The lumen through the distal end 105″ has a stepped design with a smaller-diameter first lumen segment 119A extending proximally from the open tip to a larger-diameter second lumen segment 119B, thereby defining a stepped interface 117 therebetween. The second lumen segment 119B extends proximally from the stepped interface 117 to the two slots 111A″, 111B″. Although the lumen 119A is shown to be parallel, the lumen can also be made to be slightly tapered with the major (or minor) diameter at the distal tip and the minor (or major) diameter at the stepped interface 117.

The stepped lumen design of FIG. 4 is meant to core and remove a hair follicle unit in one operation, and thus avoid the use of forceps to grasp and remove the hair follicle unit after it has been cored. More particularly, when the coring tool 101″ cores a hair follicle unit, the root of the hair follicle unit is compressed radially to some degree within the smaller-diameter first lumen segment 119A and expands slightly above the stepped interface 117. This compression can be facilitated with the aforementioned tapering of the lumen. In this manner, the root of the cored hair follicle unit becomes snagged by the stepped interface 117. The user then retracts the coring needle 101″, which removes the cored hair follicle unit from the scalp. The snagging forces provided by the stepped interface 117 maintain the hair follicle unit within the coring needle 101″ as it is retracted, thereby allowing the cored hair follicle unit to be effectively and efficiently removed from the scalp. Such operations are schematically depicted in the sequence of FIGS. 5A-5C. In these figures, the hair follicle unit to be cored (labeled 125) includes one or more hairs 126 (3 shown) and root 127 within a section of scalp 128. The coring needle 101″ of FIG. 4 is placed over the hairs 126 and cored into the scalp 128 (FIG. 5B). The stepped interface 117 snags the root 127 of the hair follicle unit. When the coring needle 101″ is withdrawn, the hair follicle unit 125 is removed from the scalp as best shown in FIG. 5C.

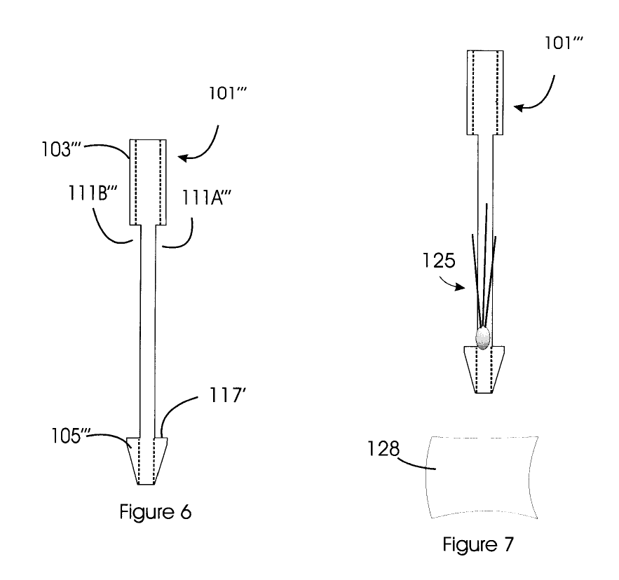

A coring needle 101′″ according to a fourth embodiment of the invention is shown in FIG. 6. The coring needle 101′″ includes a hollow tubular member 103′″ with two slots 111A′″, 111B′″ that are disposed opposite one another in the annular wall of the tubular member 103′″. Preferably, the slots 111A′″, 111B′″ each have a major dimension that extends parallel to the central axis of the tubular member 103′″ and a minor dimension that extends substantially orthogonal relative to the central axis of the tubular member 103′″ as is evident in FIG. 6. Preferably, the distal end 105′″ is sharpened, while the proximal end 101′″ is blunt. The tubular member 103′″ is constructed such that it is substantially rigid in response to the forces applied thereto during use in coring a hair follicle unit. The offset between the opened distal tip and the distal end surfaces of the slots 111A′″, 111B′″ can be shorter than the other embodiments described herein and is preferably on the order of 5.0 mm to 10.0 mm. The stopper ring can be omitted. In the configuration of FIG. 6, the distal end surfaces of the slots 111A′″, 111B′″ provide a stepped interface 117′ that snags the root of the cored hair follicle unit in a manner similar to the stepped interface 117 of FIG. 4 as described above. When the coring needle 101′″ is withdrawn, the hair follicle unit 125 is removed from the scalp as best shown in FIG. 7. In this manner, the coring needle of FIG. 6 cores and removes a hair follicle unit in one operation, and thus avoids the use of forceps to grasp and remove the hair follicle unit after it has been cored.

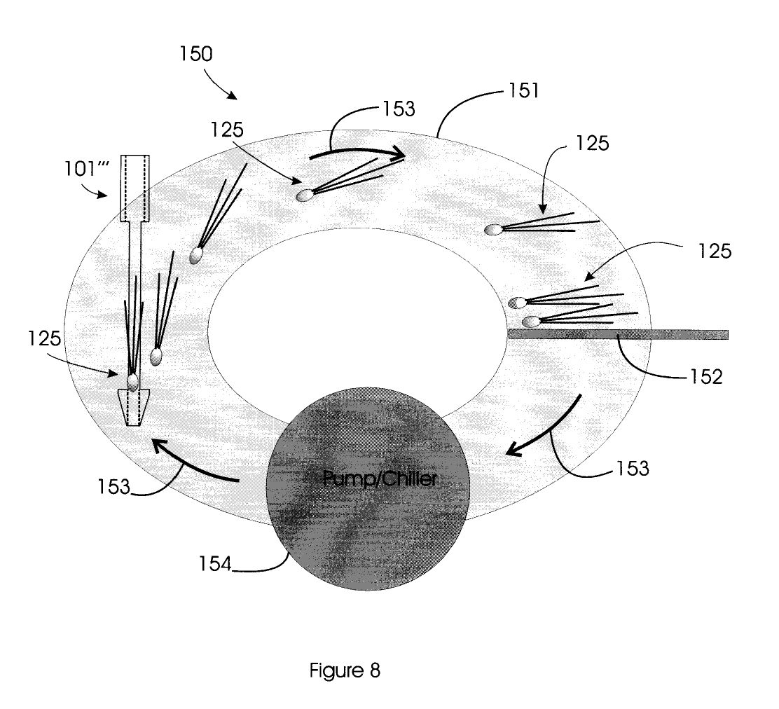

Upon withdrawal of the coring needle 101′″ from the scalp, the hair follicle unit snagged in the coring needle 101′″ can be placed in a flow loop that removes the hair follicle unit and maintains it cold. As shown in FIG. 8, the flow loop 150 includes a fluid circulation loop 151 filled with cold saline that is circulated in the directions of arrows 153 preferably by pump/chiller 154. Other liquids (e.g., Freon) suitable for maintaining hydration of hair follicle units can be used. The circulating fluid is chilled by the pump/chiller 154. A filter 152 is disposed within the circulation loop 151. The filter 152 is sized to capture hair follicle units that are carried by the circulating fluid. When the coring needle 101′″ containing a hair follicle unit 125 is placed in the fluid circulation loop 151, the saline fluid flow causes the hair follicle unit 125 to move out of coring needle 101′″ into the circulation loop 151 where it is captured in filter 152. The cold fluid solution maintains the captured hair follicle units cold and hydrated for subsequent transplantation. In this manner, the physician performing the hair coring need only place the needle tip in the flow loop 150 and the hair is automatically removed and maintained cool and hydrated. Similar operations can be carried out with the coring needle of FIG. 4.

Hair follicle units cored by the coring tool of FIGS. 1-3 and subsequently removed from the scalp can be kept cool and hydrated by the flow loop 150 of FIG. 8.

Although the coring needles of FIGS. 4 and 6 can function as depicted above, it will be appreciated that such devices can be used in different ways. For example, in the event that a hair follicle unit does not separate from the scalp by withdrawal of the coring needle, a finger can be placed in the slot to hold the hair relative to the needle slot and thereby enable the hair follicle unit to be removed with the coring needle. Alternatively, a pivoting cam or other suitable hair retaining element can be used in lieu of a finger. In this case, the hair retaining element is secured to the coring needle and the hair is forced under the hair retaining element to aid in gripping the hair during the removal procedure.

There have been described and illustrated herein several embodiments of a tool (and parts thereof) for coring a portion of one or more hair follicles and a method of operating such tool for autologous hair transplantation. While particular embodiments of the invention have been described, it is not intended that the invention be limited thereto, as it is intended that the invention be as broad in scope as the art will allow and that the specification be read likewise. Thus, while particular dimensions and materials have been disclosed, it will be appreciated that other dimensions and materials can be used as well. In addition, while particular applications of the tool have been disclosed for autologous hair follicle harvesting of the scalp, it will be understood that the tool can readily be used for hair follicle harvesting in other areas of the body or from a strip (using the traditional strip method). Moreover, while particular mechanisms have been disclosed that are capable of manually adjusting the needle depth of the tool, it will be appreciated that other mechanisms could be used as well. It will therefore be appreciated by those skilled in the art that yet other modifications could be made to the provided invention without deviating from its spirit and scope as claimed.

What is claimed is:

1. An apparatus for coring out a portion of at least one hair follicle from a patient's body comprising:
an elongate hollow tubular member having a distal end opposite a proximal end, an opened distal tip, and at least one cut-out through an annular wall of the hollow tubular member, said at least one cut-out disposed between the distal end and proximal end, whereby said cut-out allows for removal of debris that moves it way up the hollow tubular member from the opened distal tip.

2. An apparatus according to claim 1, wherein:
the distal end of the tubular member is sharpened.

3. An apparatus according to claim 1, wherein:
the tubular member is substantially rigid in response to forces applied thereto in coring out a portion of one or more hair follicles from a patient's body.

4. An apparatus according to claim 1, wherein:
the tubular member has an outer diameter in the range between 0.7 mm and 1.5 mm and has an inside diameter in the range between 0.3 mm and 1.2 mm.

5. An apparatus according to claim 1, wherein:
the open distal tip has defines an opening with a diameter in the range between 0.5 mm and 1.2 mm.

6. An apparatus according to claim 1, further comprising:
a part projecting radially outward from said hollow tubular member, said part disposed between said opened distal tip and said at least one cut-out.

7. An apparatus according to claim 6, further comprising:
a first ring-shaped member that is slipped concentrically over said distal end and that rests against said part.

8. An apparatus according to claim 6, further comprising:
at least one other ring-shaped member that is slipped concentrically over said distal end and that rests against said first ring-shaped member.

9. An apparatus according to claim 6, wherein:
said part is offset from said opened distal tip at a distance less than 10 mm.

10. An apparatus according to claim 1, wherein:
the hollow tubular member is realized from a material selected from the group including stainless steel, a titanium nitride coating, a zirconium coating, and a diamond coating.

11. An apparatus according to claim 1, wherein:
the tubular member has a central axis, and the cut-out has a major dimension that extends parallel to said central axis and a minor dimension that extends radially with respect to said central axis.

12. An apparatus according to claim 1, further comprising:
two cut-outs disposed opposite one another through an annular wall of the hollow tubular member.

13. An apparatus according to claim 1, wherein:
the distal end of said hollow tubular member includes a stepped interface that is adapted to snag a root of a hair follicle unit cored by the hollow tubular member upon its withdrawal away from the patient's body.

14. An apparatus according to claim 13, wherein:
the stepped interface is defined by the lumen of the hollow tubular member, said lumen having a smaller-diameter first segment that extends to a larger-diameter second segment.

15. An apparatus according to claim 13, wherein:
the stepped interface is defined by a distal end surface of the at least one cut-out.

16. An apparatus according to claim 13, wherein:
the stepped interface is offset from said opened distal tip at a distance less than 10 mm.

17. An apparatus for maintaining hair follicle portions cold and hydrated, the apparatus comprising:
a fluid circulation loop carrying cold circulating fluid; and
a filter disposed within the fluid circulation loop, said filter for capturing hair follicle portions that are carried by the cold circulating fluid.

18. An apparatus according to claim 17, further comprising:
an electric pump for circulating the cold circulating fluid about the fluid circulation loop.

19. An apparatus according to claim 17, further comprising:
a chiller for chilling the cold circulating fluid.

20. An apparatus according to claim 17, wherein:
the circulating fluid comprises saline.

21. An apparatus according to claim 17, further comprising:
the hollow tubular member of claim 1, wherein the circulating fluid is sufficient to remove cored hair follicle portions from said hollow tubular member.

22. An apparatus for coring skin tissue from a patient's body comprising:
an elongate hollow tubular member having a distal end opposite a proximal end, an opened distal tip, and at least one cut-out through an annular wall of the hollow tubular member, said at least one cut-out disposed between the distal end and proximal end, whereby said cut-out allows for removal of debris that moves it way up the hollow tubular member from the opened distal tip.

23. An apparatus according to claim 22, wherein:
the distal end of the tubular member is sharpened.

24. An apparatus according to claim 22, wherein:
the tubular member is substantially rigid in response to forces applied thereto in coring out the skin tissue from the patient's body.

25. An apparatus according to claim 22, wherein:
the tubular member has an outer diameter in the range between 0.7 mm and 1.5 mm and has an inside diameter in the range between 0.3 mm and 1.2 mm.

26. An apparatus according to claim 22, wherein:
the open distal tip has defines an opening with a diameter in the range between 0.5 mm and 1.2 mm.
